(19) United States
(12) Patent Application Publication
(10) Pub. No.: US 2012/0035583 A1
(43) Pub. Date: Feb. 9, 2012

(54) MULTIMODE NEUROBIOPHYSIOLOGY PROBE

(76) Inventor: Jehuda Peter Sepkuty, Redmond, WA (US)

(21) Appl. No.: 13/276,186

(22) Filed: Oct. 18, 2011

Related U.S. Application Data
(63) Continuation-in-part of application No. 12/381,999, filed on Mar. 19, 2009.

(51) Int. Cl. A61B 5/00; A61F 7/12

(52) U.S. Cl. 604/503; 600/301; 607/113

(57) ABSTRACT

Deep Brain Stimulation (DBS) is taking off and will be part of the main treatment for brain diseases such as movement disorders, epilepsy, psychiatric diseases and many others. There is a need for more sophisticated devices that can do more in one penetration, not just stimulate. Once there is a probe in the brain, it is used for multiple passive measurements, without harming the brain further. It provides better understanding the brain and real time closed loop improved treatment. An apparatus and method are disclosed, which allow simultaneous monitoring of multiple parameters inside the human brain, such as: pH, temperature, pressure, seizure activity (EEG), degree of metabolism, oxygen tension in the brain, degree of excitotoxicity and others. The ability to measure those parameters during treatment and stimulation procedures makes the difference between success and failure of the patient.

### CROSS REFERENCE TO RELATED APPLICATIONS

This patent application is a continuation-in-part of U.S. patent application Ser. No. 12/381,999, Pub. No. 20100241100 filed Mar. 19, 2009.

### FIELD OF INVENTION

This invention relates generally to the field of neuroscience, biotechnology and medical instrumentation, and particularly to molecular sampling, delivery and characterization methods applied in conjunction with optical, electromagnetic or electrochemical interrogation or excitation by means of a minimally-invasive probe at a designated site in the brain.

### BACKGROUND

U.S. Pat. No. 6,584,335 to Hans-Peter Haar describes an end-sealed hollow needle having a permeable area allowing size-limited fluid-borne molecules to be coupled via evanescent field effects through a semi-permeable coating to an optical fiber or waveguide positioned in the needle cavity. This allows optical interrogation by quantum-cascade laser-excited multiple-wavelength attenuated total reflectance spectroscopy (ATR) in the 7 to 13-micron wavelength region. This enables detection and quantification of blood glucose concentration, which, in principle, might be used to control the administration of insulin through the interior of the hollow needle surrounding the optical fiber. The efficacy of this device is dependent on unobstructed function of the permeable area of the hollow needle and on the stability of the evanescent-field coupling efficiency of the semipermeable coating of the optical fiber or light-guide; this is subject to variability with temperature and requires probe temperature measurement and heating control in order to maintain function. Another confounding effect on the ATR analysis is the possibility of fouling the semi-permeable membrane with a local concentration of small molecules or an adherent fluid-borne substance, thereby aliasing the spectral data.

US2007/0142714A1 to Daniel L. Shumate describes a needle containing bundled microtubes and optical sensing fibers. Therapeutic fluids may be delivered and extracted through microtubes by pulsatile micro-pumps. Temperature, pH and PO2 may be measured by separate fibers, which may or may not have chemical-sensing or temperature-sensing coatings. This device has no means of sample particulate or molecular size selectivity, and no means for concentration or amplification of the desired analyte(s). Target applications include tumor diagnostics, orthopedic joint and back surgery, and opthalmic surgery. Opthalmic probes are also referenced in U.S. Pat. No. 5,643,250 and U.S. Pat. No. 6,520,955.

There is a continuing need in the field of deep tissue treatment, and in particular, intracranial treatment, in improvements of the inserted probes aiming accuracy of the insertion and avoidance of injury, while retaining ease-to-use and efficiency. There is a need for more sophisticated devices that can do more in one penetration, not just stimulate. Once there is a probe in the brain, using that for multiple passive measurements, without harming the brain further, is a great opportunity to better understand the brain and provide real time closed loop improved treatment.

There is also a need to reduce a number of instruments which penetrate the tissue, especially the brain, to minimize the invasiveness.

Neurotrauma, the so-called “silent epidemic”, is the main cause of mortality and disability in the population under 40 years old. Wars, Motor vehicle accidents and other trauma are the main causes of these injuries. It is also the leading cause of years of productive life loss. Neurotrauma has predilection for young working males between 15 and 30 years old and a notorious inverse relationship with family incomes. Regarding mortality, the study stated that it was near 1% for minor injury, 18% for mild, and 48% for severe head injury.

### SUMMARY OF THE INVENTION

The invention is a system with multimodal probe for applications in neuroscience research and clinical diagnostics. Intended for use in various procedures in the brain, the device provides a minimally invasive means for the brain function monitoring while performing the treatment.

A single probe lowered in the brain accommodates at least two wires providing information about the brain living signs. Certain active treatment or interference can be performed at the same time. A set of measuring units connected to the probe allows monitoring the treatment in real time thus improving the outcome.

The monitoring characteristics include: intracranial pressure, temperature, pH, EEG, Oxigen tention, and many others. The active interrogation includes the drug delivery, laser pulse stimulation and others.

Combinations of two or more of these techniques, applied simultaneously or sequentially at the same site allows dramatically improve the treatment and save lives.

### BRIEF DESCRIPTION OF THE DRAWINGS

FIG. 1 depicts a first embodiment of the invention for monitoring EEG, temperature, and acidity in the brain, while performing a treatment.

FIG. 2 shows the proposed system configuration adapted for the case of a severe head trauma.

FIG. 3 shows real measurement results obtained both from a surface probe (a) and from a probe inserted into the brain (b).

FIG. 4 shows a schematic approach to the probe structure.

### DETAILED DESCRIPTION OF THE PREFERRED EMBODIMENT

The device structure shown in FIG. 1 comprises a probe with a multi-port manifold body 1. The probe may be inserted inside a tissue. In the preferred embodiment, it is implementation to control brain functioning during various intracranial abnormalities: head trauma, epilepsy, stroke, Parkinson disease and other. The probe is inserted in the human brain such as shown in FIG. 1.

The manifold body 1 may be fabricated from stainless steel, titanium, ceramic, glass, acetyl (or some other polymer). The tubing must also be a biocompatible material, not necessarily the same as that of the manifold body. Appropriate material selection allows fabrication of probes which are compatible with MRI or other imaging procedures.

The functional part of the device is the microtube 2, typically a section of stainless steel or titanium hypodermic tubing (typically 100 to 300-micron internal diameter and having a typical working length from 2 mm to 100 mm) which is inserted into the tissue site of interest.

The tube is wide enough to accommodate multiple wires transmitting signals to and from the tissue. By saying “wire” we do not limit ourselves by just metal wires to transmit electrical signals. In our case, “wire” means any kind of connecting links: optical waveguides, metal wires, tubes for liquid delivery and extraction or any other.

In particular, the invention provides improvement to current procedures of clinical diagnostics and treatment in cases of a severe head trauma in military operation and civil accidents. The final common pathway for death and permanent disability in head injuries and brain disease is usually increased intracranial pressure, but there are multiple other parameters which are important to monitor to guide treatment during the critical period.

All neuro surgeons and head trauma experts would agree that the more parameters can be measured and monitored simultaneously, the better it would be for the patient in terms of ability to understand and respond as fast as possible in the critical period.

Parameters such as: PH, temperature, pressure, seizure activity (EEG), degree of metabolism, oxygen tension in the brain, degree of excitotoxicity, blood flow, upregulation/downregulation of specific neurotransmitters are all crucial to evaluate the situation and respond by stabilizing and offering the right treatment. The ability to monitor pressure, temperature, Ph, EEG recording, optical measurement of oxygen tension, electrochemical measurement of specific transmitters, all at the same time is invaluable and may make the difference between success and failure of treatment.

The probe is shown in FIG. 1 with the microtube 2 wide enough in diameter, with multiple monitoring wires through the same shaft allows multimodality and simultaneous monitoring of multiple parameters.

The simultaneous fast and reliable measurement of multiple parameters, not affecting one measurement by the simultaneous measurement and monitoring of the others is unique, innovative and different from the current existing probes. The same probe, then can be used for multiple type treatments after the passive measurements such as: lowering pressure, cooling, changing PH, stimulating to control seizure activity, increasing oxygen tension and delivering local medications, which could be done at least in part simultaneously interchanging between passive measurements of treatment effects and treatment in real time.

We demonstrate in FIG. 1 the arrangement of units 3-7, connected to the microtube 2, in a case of epileptic seizures/activity or suspicion of that regardless of brain trauma. The electroencephalograph 3 performs EEG recording by sensors attached to the outer surface of the microtube 2. In one embodiment, the probe has metal electrode rings 3 mm apart on the outside of the microtube connected to tiny wires connectable to a cable and EEG machine 3 or downloadable to a computer chip and transferred to EEG machine able to record EEG or apply stimulation between two specific electrodes chosen as anode and cathode. This assures recording from different depth of the brain dependent on electrode placement in relation to brain depth.

The temperature measuring unit 4 (FIG. 1) measures the temperature by sensing from a dithermic material conducted through wire to the scope/computer.

Deoxyglucose vs. oxyglucose concentration indicates metabolism. It is important to determine regions of increased metabolism, since seizure activity tend to have higher metabolic demand and this will be additional independent proof of seizure activity with further localization data. The spectrophotometer 5 in FIG. 1 performs such measurement.

Additional measuring units, indicated as 6 in FIG. 1, may provide additional information about the tissue. For example, the unit 6 may be connected to an electrochemical sensor positioned at the end of the microtube 2. The electrochemical sensor is indicative of glutamate in extracellular space or, alternatively, GABA in extracellular space. The functioning of unit 6 is not limited by this description, it can be any other parameter measurement, which is helpful in diagnostics or treatment of the patient.

In another embodiment, the unit 6 provides pH measurement. PH is measured as in any biologic lab by a sensor sensitive to H+ ion concentration translated to electrical measurement and calibrated to present as numbers reflecting acidity/alkalinity: bellow 7 reflecting acidity and above 7 reflecting alkalinity.

In yet another embodiment, the unit 6 measures an intracranial pressure, which is crucial to monitor allowing treatment interference to keep at the right level.

The unit 7 is connected with the interrogated tissue for treatment or stimulation. The arrow 8 shows the direction of the signal coming from the unit 7 toward the patient brain.

Various types of anti-seizure actions can be implemented. For example, anti seizure medications may be delivered to the interrogated volume. In another embodiment, a cooling is provided helping to abort the seizures. In yet another embodiment, measures affecting metabolism are implemented. In the case of low pH (acidosis) one can modify pH by modifying ventilation rate (pt is comatose, intubated and ventilated by a machine. Increasing respiratory rate will decrease PCo2 and decreasing respiratory rate on the ventilator will cause increase of PCo2 which in turn affects acidity of the brain tissue: this is the common way of modifying PH in the comatose patient in critical care setting.

In the case of interfering to treat seizures: local antiepileptic seizures (maximum effect with less systemic side effects), concomitant stimulation through same contacts that passively recorded the EEG, blocking excitatory receptors may be implemented by the unit 7.

Having all these monitored may help treat seizures and maybe even predict seizures in the acute phase where immediate treatment is crucial. Recent studies the use of tetrodes (four depth electrodes) in rat brain and applying sophisticated mathematical algorythms allowing to predict seizures and treat preventively.

Now let us consider another example of the system application. It is hard to overestimate the importance of immediate help in case of severe head trauma. Timely diagnostic and accurate response can save many lives both in military operations and in civil environment.

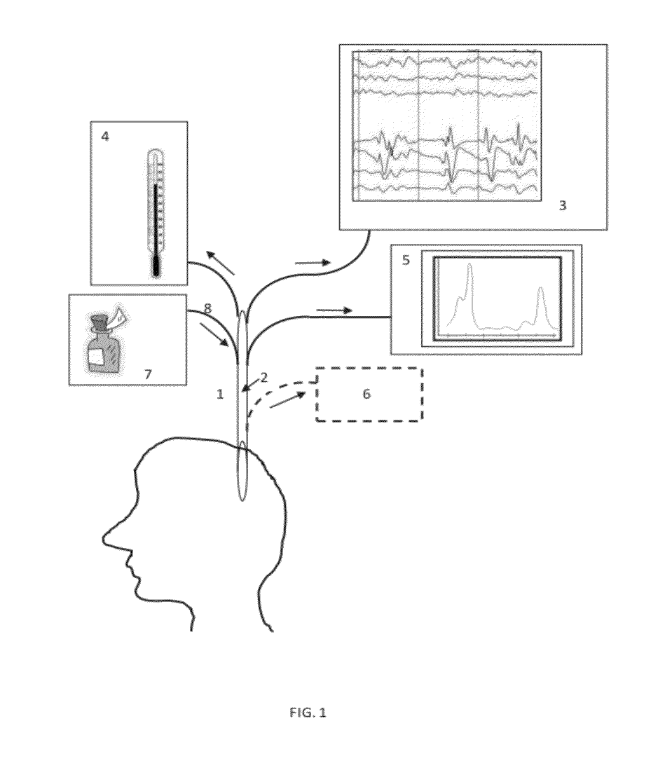

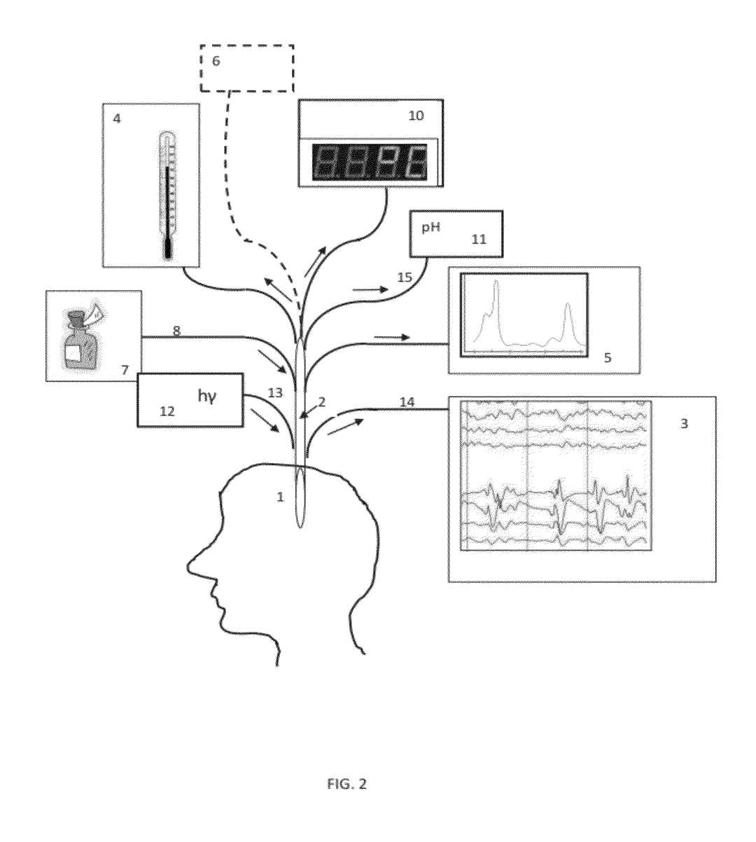

The device presented in FIG. 2 is adapted for the case of a severe head trauma (penetrating or closed). In such cases, it is beneficial to monitor at least the following brain parameters:

1. Pressure

2. Temperature sensor

3. PH

4. EEG from depth

5. Oxygen tension (partial pressure)

6. Oxigenated hemoglobin vs. deoxygenated

7. NMDA glutamate receptor changes

In the preferred embodiment the intracranial pressure is measured by a piatzo electric sensor, and the measured data is displayed on a monitor 10.

The acidity measurement is performed by pH unit 11 as previously explained in paragraph 30.

Other measuring units 3-6 allow monitoring of various necessary parameters of the brain living signs listed above.

FIG. 2 also shows two units 7 and 12 for active interference with the brain functioning.

The unit 7 includes a pump system, which delivered or extract fluid from the tissue site proximal to the end of the microtube 2 via a fluid delivery tube 8. It can be done similarly to the procedure describes in of U.S. Pat. No. 7,608,064 and shown in FIG. 6 of that patent, which demonstrates the depth probe for intracranial treatment allowing delivery of a drug to a targeted place of the tissue.

Pressure measurement by unit 10 allows continuous pressure reading, and the doctor is able to interfere by high osmolarity glucose (manitol) delivery, hyperventilation procedure and steroids delivery via tube 8 thus lowering swelling and therefore pressure.

Temperature measuring by unit 4 and simultaneously cooling locally by some peltier device or local scalp cooling allows reducing swelling.

PH sensor attached to the acidosis of the brain (low PH) measuring unit 11 allow adjusting the PH through changes of rate of ventilation (via tube 8) affecting PCO₂ and indirectly PH or use locally CO₂ to increase/decrease PCO₂.

EEG is monitored in the unit 3, and one can see and conclude that seizures need to be treated. No evidence in literature for preventative seizure treatment can help so proving seizure activity is crucial. Predicting seizures though would be extremely beneficial once worked out further. The outside of the metal microtube 2 can have electrodes sensing depth EEG while the inside of microtube used to introduce the wires to measure the other modalities.

Oxigen partial pressure/concentration as well as oxy and deoxyhemoglobin can tell us how oxiganated the injured tissue is and in response increasing oxygen or giving some agent to shift more deoxyganated to oxygenated hemoglobin would be helpful.

Measuring glutamate concentration extracellularly by an electrochemical sensor vs microdyalisis can dictate medications which are blocking glutamate NMDA receptors or choosing a local drip of NMDA glutamate blocker.

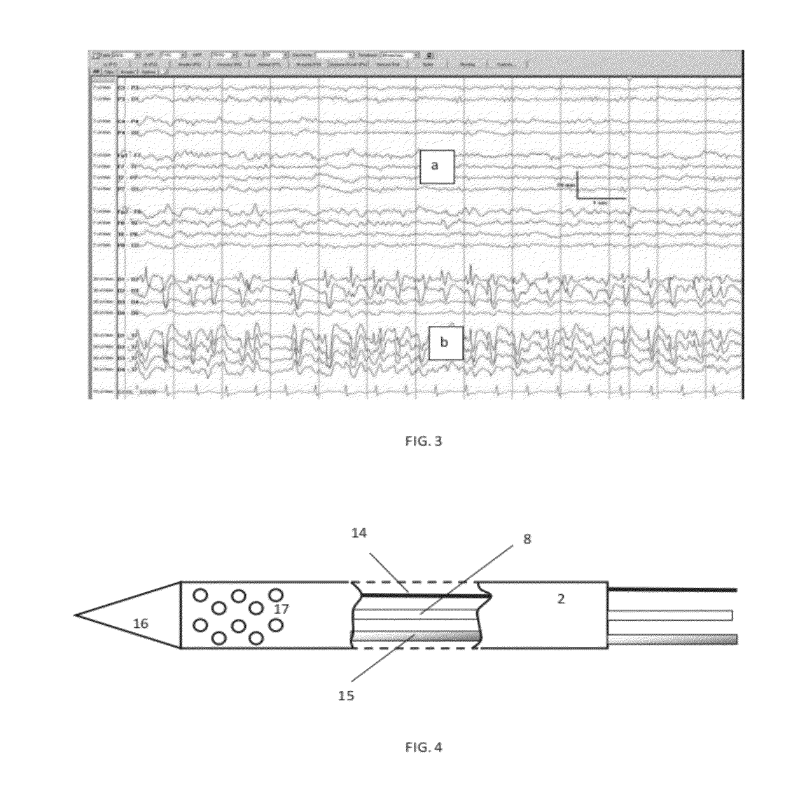

FIG. 3 shows real measurement results obtained both from a surface probe (a) and from a probe inserted into the brain (b). This example of a patient with subarchnoid hemorrhage, where the probe positioned on scalp (upper channels) has no indication of seizure activity while minielectrodes penetrating brain by 3 mm show severe seizure activity (b). Subarachnoid hemhorage is common in severe brain trauma. This figure demonstrates importance of a probe lowered inside the brain to get information about the brain functioning.

In yet another embodiment, the system includes other stimuli for active and passive interference with the brain functioning. FIG. 2 shows a light source 12 with an optical fiber 13 coupled to it. The light source is constructed and arranged to emit a laser beam of visible or infra-red radiation. Laser light is coupled into the fiber 13, delivered to the tissue via the microtube 2. The light detector is optically coupled to the fiber detect photons of radiation reflected back from the tissue. The processor is operatively coupled to the light source and detector and is adapted to determine an optical property of the biological tissue of interest based on the changes between the introduced and detected radiation. For example, the measurement can be performed such as described in U.S. patent application Pub. No. US 2009/0030327.

Alternatively a small video camera may be attached at the end of the fiber 3 (not shown in the FIG. 2). The camera translates the image of the tissue to a monitor, where an operator can distinguish various types of tissue and see their characteristics.

The optical interrogation may be done directly from the tissue or fluid by any of the well-known spectroscopy technologies in an optical spectroscopy system.

Yet in another embodiment, a chemical sensor coating at the tip of the optical fiber 13 is deposited, such as, for example, a Ruthenium Dioxide coating whose fluorescence properties are responsive to Oxygen concentration.

Alternatively, the optical fiber tip may also be coated with an immobilized optical reporter material which reacts to a target analyte (neurotransmitter or other protein) molecule; this reaction may occur either directly to the target analyte or indirectly to a binding agent specific to the target analyte.

In yet another embodiment, the tissue may be actively stimulated by optical pulses delivered via the fiber 13. Optical stimulation can be another stimulation, same as electrical stimulation used to treat seizures, etc. (DBS), but may be more local and less spreading through axons, i.e. less likely to cause a seizure and may allow using in conjunction with electrical stimulation without exceeding allowed current density delivery to brain tissue, while adding to treatment effect.

For the diagnostic and treatment of patients with stroke the probe can check if a blood vessel obstructed by optical way and respond with TPA (chemical used to dissolve clots). Also an ultrasound technology is used to help break off the clot faster using mechanical energy of the ultrasound together with TPA. All those types of treatment may be administered via the probe disclosed in the present invention. The probe in this case gets into a blood vessel rather than brain tissue or penetrating brain and gets within it a blood vessel.

in yet another embodiment, the present invention is used for brain tumors diagnostic by using a small video camera, looking at the tumor, its vascularity and by interrupting its vascularity causing some shrinkage which can help make surgery easier and local chemotherapy treatment again can save a lot of very bad systemic side effects (nausea, vomiting, hair loss etc.).

Various types of probe configurations may used for the technology described above. The preferred embodiment is disclosed in more details in the co-pending parent U.S. patent application Ser. No. 12/381,999, filed Mar. 19, 2009. Here we illustrate the main features of the probe in FIG. 4. An opening in the middle of the microtube 2 is made to show the wires inside it, it is not present in the real probe.

The microtube 2 must be wide enough to accommodate a number of wires (at least two, but the more the better). As an example, FIG. 4 shows electrical wire 14 for EEG monitoring, an optical fiber 15 for pH measurement and a drug delivery tube 8. The microtube may have various types of ending to facilitate the probe penetration into the tissue and provide minimal damage to it. FIG. 4 shows a tapering end 16, which is one of the possible solution, but the scope of solutions is not limited to this one.

The microtube may optionally have a set of apertures 17 for suction of a liquid surrounding the probe and its further delivery to the measuring unit. The aperture may also serve for the drug delivery to the tissue.

One or more other types of wires for electrical, optical, fluidic, chemical or biological parameters measured can fit into the microtube 2. It allows providing a treatment and monitoring simultaneously, which improves the outcome of the treatment.

It is another object of the present invention to provide a set of probes that were described above thus monitoring large areas of the brain with multiple probes.

While embodiment of the present invention has been described above, it should be understood that it has been presented by way of example only, and not limitation. Thus, the breadth and scope of the present invention should not be limited by the above-described exemplary embodiment, but should be defined only in accordance with the following claims and their equivalents.

The previous description of the preferred embodiment is provided to enable any person skilled in the art to make or use the present invention. While the invention has been particularly shown and described with reference to preferred embodiment thereof, it will be understood by those skilled in the art that various changes in form and details may be made therein without departing from the spirit and scope of the invention.

What is claimed is:

1. An apparatus for a patient diagnosis, monitoring and treatment, comprising:
a probe including a tube, the tube being inserted in a tissue; the tube diameter is big enough to accommodate at least two wires to fit through it;
at least two wires being connected to at least two units performing measurement of different parameters of the tissue; the wires connecting those units with an interrogated point inside the tissue; and
at least two units performing the measurements simultaneously.

2. The apparatus of claim 1, wherein the tissue is human brain.

3. The apparatus of claim 2, wherein a first wire is connected to an electroencephalograph to perform an EEG recording.

4. The apparatus of claim 3, wherein a second wire performs a temperature measurement.

5. The apparatus of claim 4, wherein a third wire performs a cooling of the interrogated point inside the tissue to abort seizures, while the temperature measurement results indicate when to stop the cooling.

6. The apparatus of claim 2, further comprising a third wire, wherein the third wire actively interfere the tissue at the interrogated point simultaneously with the measurements via a first and a second wires.

7. The apparatus of claim 6, wherein an anti-seizure medication administered via the third wire.

8. The apparatus of claim 6, wherein an optical pulse stimuli is directed to the tissue via the third wire.

9. The apparatus of claim 2, wherein at least one of the wires is an optical fiber and at least one wire in a metal wire.

10. The apparatus of claim 2, wherein at least two measurements performed by the apparatus are selected from EEG, temperature, intracranial pressure, pH, oxygen concentration, oxygen tension, deoxyglucose vs. oxyglucose, glutamate concentration, and GABA measurement.

11. An apparatus for a patient diagnostic and treatment, comprising:
a probe including a tube, the tube being inserted in a tissue; the tube diameter is big enough to accommodate at least three wires to fit through it;
at least three wires being connected to at least three units performing measurements of different parameters of the tissue; the wires connecting those units with an interrogated point inside the tissue; and
at least three units performing the measurements simultaneously.

12. The apparatus of claim 11, wherein the tissue is human brain.

13. The apparatus of claim 12, wherein a first unit measures intracranial pressure to determine the swelling condition, and a second unit measures a temperature, and a third unit measures an acidity (pH).

14. The apparatus of claim 13, further comprising a four unit measuring simultaneously a parameter, selected from an encephalogram (EEG) in the tissue depth to detect an appearance of seizures; an oxygen tention; an oxigenated vs. deoxigenated hemoglobin or a glutamate concentration.

15. The apparatus of claim 13, further comprising a fourth wire carried out through the same tube; wherein an active interference with the tissue is performed via the fourth wire.

16. The apparatus of claim 15, wherein the interference is a drug delivery or an optical pulse stimuli or an electrical pulse stimuli.

17. The apparatus of claim 15, wherein the interference is administering locally CO2 to change PCo2 thus adjusting pH to a normal level; the CO2 being directed to the interrogated point via the same tube.

18. The apparatus of claim 10, wherein the three measurements are selected from EEG, temperature, intracranial pressure, pH, oxygen concentration, oxygen tension, deoxyglucose vs. oxyglucose, glutamate concentration, and GABA measurement.

19. A method of a patient diagnostic and treatment, comprising:
lowering a probe in the patient brain; the probe being minimally damaging for the patient;
performing the patient treatment via a wire passed through the probe;
simultaneously measuring at least two parameters of the brain via wires passed through the probe;
determining a dosage of the treatment basing on results of the both measurements.

20. The method of claim 19, wherein one of the measuring parameters is EEG.
